(19) United States
(12) Patent Application Publication
(10) Pub. No.: US 2010/0104664 A1
(43) Pub. Date: Apr. 29, 2010

(54) TREATMENT OF CHRONIC RENAL FAILURE AND OTHER CONDITIONS IN DOMESTIC ANIMALS: COMPOSITIONS AND METHODS

(76) Inventors: Allison Wren, Reno, NV (US); Roy Graham, Incline Village, NV (US); Randy McElreath, Reno, NV (US); Michael Molnar, Summerfield, NC (US)

Correspondence Address: LERNER, DAVID, LITTENBERG,;KRUMHOLZ & MENTLIK, 600 SOUTH AVENUE WEST, WESTFIELD, NJ, 07090, US

(21) Appl. No.: 12/643,518

(22) Filed: Dec. 21, 2009

Related U.S. Application Data
(63) Continuation of application No. 11/465,442, filed on Aug. 17, 2006, now abandoned.
(60) Provisional application No. 60/709,180, filed on Aug. 17, 2005.
(60) Provisional application No. 60/721,774, filed on Sep. 29, 2005.

(51) Int. Cl. A61K 31/59; A61K 31/19; A61K 33/10; A61K 33/08; A61K 31/40; A61K 31/55; A61K 31/165; A61K 31/138; A61K 31/4422; A61K 38/00

(52) U.S. Cl. 424/687; 514/167; 424/715; 514/557; 424/690; 514/423; 514/212.07; 514/620; 514/652; 514/356; 514/8

(57) ABSTRACT

The present invention generally concerns the management of age-related diseases in domestic animals. Specifically, the present invention is directed to combination therapies for the treatment of progressive renal diseases (e.g., Chronic Renal Failure) and their accompanying secondary disease states. In a composition aspect, the present invention provides a composition comprising a phosphate binder and another pharmaceutically active ingredient. The other pharmaceutically active ingredient is selected from a group consisting of antihypertensives, calcitrol, vitamin D analogues, lipid restriction products, potassium salts, treatments for anemia and alkalization compounds.

### CROSS-REFERENCE TO RELATED APPLICATIONS

This application claims priority to U.S. Provisional Patent Application Ser. No. 60/709,180 filed on Aug. 17, 2005 and U.S. Provisional Patent Application Ser. No. 60/721,774 filed on Sep. 29, 2005, the entire disclosures of which are incorporated by reference.

### FIELD OF THE INVENTION

The present invention generally concerns the management of age-related diseases in domestic animals. Specifically, the present invention is directed to combination therapies for the treatment of progressive renal diseases (e.g., Chronic Renal Failure) and their accompanying secondary disease states.

### BACKGROUND OF THE INVENTION

Chronic Renal Failure (CRF), also called Chronic Renal Insufficiency (CRI), is defined as primary renal failure that has persisted for a prolonged period. The condition is characterized by the presence of irreversible structural lesions in the kidneys and is considered, at least in clinical cases to be progressive and, ultimately, to lead to the death of the cat or dog. Therapy is thus aimed at ameliorating the clinical signs and slowing the progress of the disease. Hyperphosphatemia is associated with declining renal function as the kidneys can no longer remove excess phosphate from the blood.

Controversy still surrounds the etiopathogenesis of progression in both dogs and cats; nevertheless, measures that slow progression will maintain the animal at a level of glomerular filtration rate at which clinical signs of renal dysfunction are tolerable. Systemic and metabolic abnormalities associated with the loss of renal function affect almost every body system and include: Hyperphosphatemia; Hyperparathyroidism; Dyslipoproteinemias; Systemic hypertension; Metabolic acidosis; Azotemia; Failure of hormone production (including erythropoietin that stimulates the production of red blood cells; and, at the end stages of the disease fluid and electrolyte balance is severely disturbed.

Renal secondary hyperparathyroidism (RHPTH) is the major complication of CRF. It is characterized by increased endogenous levels of parathyroid hormone (PTH). With progressive CRF, hyperphosphatemia occurs as the glomerular filtration rate decreases. This leads to lower serum ionized calcium concentrations. Renal synthesis of calcitriol is also reduced. Since calcitriol is involved in the homeostasis of serum calcium concentrations, decreased ionized calcium and calcitriol cause an increase in serum PTH resulting in the clinical manifestations of RHPTH. These include vomiting, dehydration, polydipsia, depression and hyperosteotic bone lesions such as face swelling which is particularly common in younger dogs.

The hypothesis that all renal diseases are inherently progressive and self-perpetuating has focused attention on adaptive changes in renal structure and function that occur whenever renal function is reduced. These glomerular adaptations to renal disease include increases in filtration rate, capillary pressure and size, and are referred to as glomerular hyperfiltration, glomerular hypertension and glomerular hypertrophy, respectively. Extrarenal changes, such as dietary phosphate excess, systemic hypertension, hyperlipidaemia, acidosis and hyperparathyroidism occur in animals with renal disease and are contributors to progression of renal disease. Emphasis in the management of companion animals with renal disease has shifted to identifying, understanding and controlling those processes that play a role in the progression from early to end-stage renal failure.

Such progressive renal diseases are oftentimes correlated with age in domestic animals. For instance 70 percent of dogs over 5 years old and 30 percent of cats over 10 years old show the beginning signs of CRF. Accompanying problems or disease states arise with further aging.

There is accordingly a need in the art for therapies that will address progressive renal diseases in domestic animals. There is a further need in the art for treatments that will delay or even prevent the onset of such age-related diseases, which will substantially increase the quality of life for the animals. Those are objects of the present invention.

### SUMMARY OF THE INVENTION

The present invention generally concerns the management of age-related diseases in domestic animals. Specifically, the present invention is directed to combination therapies for the treatment of progressive renal diseases (e.g., Chronic Renal Failure) and their accompanying secondary disease states.

In a composition aspect, the present invention provides a composition comprising a phosphate binder and another pharmaceutically active ingredient. The other pharmaceutically active ingredient is selected from a group consisting of antihypertensives, calcitrol, vitamin D analogues, lipid restriction products, potassium salts, anemia treatments and alkalization compounds.

In a kit aspect, the present invention provides a kit for the management of age-related diseases in domestic animals (e.g., treating CRF). The kit includes: a container, wherein the container includes a composition, and wherein the composition comprises a phosphate binder and another pharmaceutically active ingredient, and wherein the other pharmaceutically active ingredient is selected from a group consisting of antihypertensives, calcitrol, vitamin D analogues, lipid restriction products, potassium salts, anemia treatments and alkalization compounds; and, instructions related to how the composition should be administered to a domestic animal.

In a method aspect, the present invention provides a method of managing age-related diseases in domestic animals (e.g., treating CRF). The method includes the following step: administering a composition to a domestic animal, wherein the composition comprises a phosphate binder and another pharmaceutically active ingredient, and wherein the other pharmaceutically active ingredient is selected from a group consisting of antihypertensives, calcitrol, vitamin D analogues, lipid restriction products, potassium salts, anemia treatments and alkalization compounds.

### BRIEF DESCRIPTION OF THE DRAWINGS

FIG. 1 shows an X-ray diffraction scan of a compound made according to Example 1, as compared to a reference standard.

FIG. 2 shows an X-ray diffraction scan of a compound made according to Example 2, as compared to a reference standard.

### DETAILED DESCRIPTION OF THE INVENTION

The present invention generally concerns the management of age-related diseases in domestic animals. Specifically, the present invention is directed to combination therapies for the treatment of progressive renal diseases (e.g., Chronic Renal Failure) and their accompanying secondary disease states.

Compositions of the present invention typically include a phosphate binder and at least one other pharmaceutically active compound. Rare earth compounds, hydrophilic anion exchange resins, calcium salts and aluminum salts are typical classes of phosphate binding compounds.

Where the compound is a rare earth compound, it is usually a lanthanum carbonate, lanthanum carbonate hydroxide or lanthanum oxycarbonate. Lanthanum carbonates are of the structure La₂(CO₃)₃.x H₂O, where 1≦x≦8. Preferred lanthanum carbonates are of the structure La₂(CO₃)₃.x H₂O, where 3≦x≦6, more preferably 3.5≦x≦5, and most preferably 3.8≦x≦4.5. Such compounds are discussed in U.S. Pat. No. 5,968,976, which is hereby incorporated-by-reference for all purposes.

Lanthanum oxycarbonates may be hydrated or anhydrous. A typical hydrated lanthanum oxycarbonate is La₂O(CO₃)₂.xH₂O, where 1≦x≦3; a typical anhydrous lanthanum oxycarbonate is La₂O₂CO₃. Such compounds are discussed in U.S. Pat. Appl. 2004161474, which is hereby incorporated-by-reference for all purposes.

Lanthanum carbonate hydroxides may be hydrated or anhydrous. A typical anhydrous lanthanum carbonate hydroxide is LaCO₃OH.

At the physiological pH of stomach of a cat and dog, around 3.0, the lanthanum oxycarbonates or lanthanum carbonate hydroxides exhibit a phosphate binding capacity of at least 300 mg of phosphate per gram of lanthanum compound. Most desirably, the lanthanum oxycarbonates exhibit a phosphate binding capacity of at least 400 mg PO₄/g of lanthanum compound. At the physiological pH of the upper small intestine of the cat or dog, around 8.0, the lanthanum oxycarbonates still bind as much as 20 mg phosphate/g lanthanum compound.

Hydrophilic anion exchange resins included in the compositions of the present invention are typically aliphatic amine polymers. The “amine” group can be present in the form of a primary, secondary or tertiary amine, quaternary ammonium salt, amidine, guanadine, hydrazine, or combinations thereof. The amine can be within the linear structure of the polymer (such as in polyethylenimine or a condensation polymer of a polyaminoalkane, e.g. diethylenetriamine, and a crosslinking agent, such as epichlorohydrin) or as a functional group pendant from the polymer backbone (such as in polyallylamine, polyvinylamine or poly(aminoethyl)acrylate). Such compounds are discussed in U.S. Pat. No. 6,858,203, which is hereby incorporated-by-reference for all purposes.

In one aspect, the polymer is characterized by a repeating unit having the formula:

—[CH₂CH(CH₂NR₂)]ₙ—

or a copolymer thereof, wherein n is an integer and each R, independently, is H or a substituted or unsubstituted alkyl, such as a lower alkyl (e.g., having between 1 and 5 carbon atoms, inclusive), alkylamino (e.g., having between 1 and 5 carbons atoms, inclusive, such as ethylamino) or aryl (e.g., phenyl) group.

In a second aspect, the polymer is characterized by a repeating unit having the formula:

—[CH₂CH(CH₂NR₃X)]ₙ—

or a copolymer thereof, wherein n is an integer, each R, independently, is H or a substituted or unsubstituted alkyl (e.g., having between 1 and 5 carbon atoms, inclusive), alkylamino (e.g., having between 1 and 5 carbons atoms, inclusive, such as ethylamino) or aryl (e.g., phenyl) group, and each X is an exchangeable negatively charged counterion.

One example of a copolymer according to the second aspect of the invention is characterized by a first repeating unit having the formula:

—[CH₂CH(CH₂NR₃X)]ₙ—

wherein n is an integer, each R, independently, is H or a substituted or unsubstituted alkyl (e.g., having between 1 and 5 carbon atoms, inclusive), alkylamino (e.g., having between 1 and 5 carbons atoms, inclusive, such as ethylamino) or aryl group (e.g., phenyl), and each X is an exchangeable negatively charged counterion; and further characterized by a second repeating unit having the formula:

—[CH₂CH(CH₂NR₂)]ₙ—

wherein each n, independently, is an integer and each R, independently, is H or a substituted or unsubstituted alkyl (e.g., having between 1 and 5 carbon atoms, inclusive), alkylamino (e.g., having between 1 and 5 carbons atoms, inclusive, such as ethylamino) or aryl group (e.g., phenyl).

In a fourth aspect, the polymer is characterized by a repeating unit having the formula:

—[N(R)CH₂CH₂]ₙ—

or a copolymer thereof, wherein n is an integer, and R is H or a substituted or unsubstituted alkyl (e.g., having between 1 and 5 carbon atoms, inclusive), alkylamino (e.g., having between 1 and 5 carbons atoms, inclusive, such as ethylamino) or aryl group (e.g., phenyl).

One example of a copolymer according to the second aspect of the invention is characterized by a first repeating unit having the formula:

—[N(R)CH₂CH₂]ₙ—

wherein n is an integer, and R is H or a substituted or unsubstituted alkyl (e.g., having between 1 and 5 carbon atoms, inclusive), alkylamino (e.g., having between 1 and 5 carbons atoms, inclusive, such as ethylamino) or aryl group (e.g., phenyl); and further characterized by a second repeating unit having the formula:

—[N(X)(H)(R)CH₂CH₂]ₙ—

wherein each n, independently, is an integer and R is H or a substituted or unsubstituted alkyl (e.g., having between 1 and 5 carbon atoms, inclusive), alkylamino (e.g., having between 1 and 5 carbon atoms, inclusive, such as ethylamino) or aryl group (e.g., phenyl).

In a fifth aspect, the polymer is characterized by a repeating group having the formula:

—[N(X)(R₁)(R₂)CH₂CH₂]ₙ—

or a copolymer thereof, wherein n is an integer, and each R₁ and R₂, independently, is H or a substituted or unsubstituted alkyl (e.g., having between 1 and 5 carbon atoms, inclusive), and alkylamino (e.g., having between 1 and 5 carbons atoms, inclusive, such as ethylamino) or aryl group (e.g., phenyl), and each X is an exchangeable negatively charged counterion.

In one preferred polymer according to the fifth aspect of the invention, at least one of the R groups is a hydrogen atom.

In a sixth aspect, the polymer is characterized by a repeat unit having the formula

—[CH(NR₁R₂)CH₂]ₙ—

or a copolymer thereof, where n is an integer, each R₁ and R₂, independently, is H, a substituted or unsubstituted alkyl group containing 1 to 20 carbon atoms, an alkylamino group (e.g., having between 1 and 5 carbons atoms, inclusive, such as ethylamino), or an aryl group containing 6 to 12 atoms (e.g., phenyl).

In a seventh aspect, the polymer is characterized by a repeat unit having the formula

—[CH(NR₁R₂R₃X)CH₂]ₙ—

or a copolymer thereof, wherein n is an integer, each R₁, R₂ and R₃, independently, is H, a substituted or unsubstituted alkyl group containing 1 to 20 carbon atoms, an alkylamino group (e.g., having between 1 and 5 carbons atoms, inclusive, such as ethylamino), or an aryl group containing 6 to 12 atoms (e.g., phenyl), and each X is an exchangeable negatively charged counterion.

In each case, the R groups can carry one or more substituents. Suitable substituents include therapeutic anionic groups, e.g., quaternary ammonium groups, or amine groups, e.g., primary and secondary alkyl or aryl amines. Examples of other suitable substituents include hydroxy, alkoxy, carboxamide, sulfonamide, halogen, alkyl, aryl, hydrazine, guanidine, urea, and carboxylic acid esters, for example.

The polymers are preferably crosslinked, in some cases by adding a crosslinking agent to the reaction mixture during or after polymerization. Examples of suitable crosslinking agents are diacrylates and dimethacrylates (e.g., ethylene glycol diacrylate, propylene glycol diacrylate, butylene glycol diacrylate, ethylene glycol dimethacrylate, propylene glycol dimethacrylate, butylene glycol dimethacrylate, polyethyleneglycol dimethacrylate, polyethyleneglycol diacrylate), methylene bisacrylamide, methylene bismethacrylamide, ethylene bisacrylamide, epichlorohydrin, epibromohydrin, toluene diisocyanate, ethylenebismethacrylamide, ethylidene bisacrylamide, divinyl benzene, bisphenol A dimethacrylate, bisphenol A diacrylate, 1,4 butanedioldiglycidyl ether, 1,2 ethanedioldiglycidyl ether, 1,3-dichloropropane, 1,2-dichloroethane, 1,3-dibromopropane, 1,2-dibromoethane, succinyl dichloride, dimethylsuccinate, acryloyl chloride, or pyromellitic dianhydride.

The amount of crosslinking agent is typically between about 0.5 and about 75 weight %, and preferably between about 1 and about 25% by weight, based upon the combined weight of crosslinking and monomer. In another embodiment, the crosslinking agent is present between about 2 and about 20% by weight of polymer.

In some cases the polymers are crosslinked after polymerization. One method of obtaining such crosslinking involves reaction of the polymer with difunctional crosslinkers, such as epichlorohydrin, succinyl dichloride, the diglycidyl ether of bisphenol A, pyromellitic dianhydride, toluence diisocyanate, and ethylenediamine. A typical example is the reaction of poly(ethyleneimine) with epichlorohydrin. In this example the epichlorohydrin (1 to 100 parts) is added to a solution containing polyethyleneimine (100 parts) and heated to promote reaction. Other methods of inducing crosslinking on already polymerized materials include, but are not limited to, exposure to ionizing radiation, ultraviolet radiation, electron beams, radicals, and pyrolysis.

Examples of preferred crosslinking agents include epichlorohydrin, 1,4 butanedioldiglycidyl ether, 1,2 ethanedioldiglycidyl ether, 1,3-dichloropropane, 1,2-dichloroethane, 1,3-dibromopropane, 1,2-dibromoethane, succinyl dichloride, dimethylsuccinate, toluene diisocyanate, acryloyl chloride, and pyromellitic dianhydride.

Where the phosphate binding compound is a calcium salt, it is typically calcium acetate or calcium carbonate. Where it is an aluminum salt, the compound is typically aluminum hydroxide.

The other pharmaceutically active compound that is combined with the phosphate binder in a composition of the present invention is typically selected from the following group: antihypertensives (e.g., ACE inhibitors, beta blockers, and calcium channel blockers); calcitrol and vitamin D analogues; protein and lipid restriction products that inhibit absorption of lipids in the gut; potassium salts; anemia treatments such as Epogen®; alkalization compounds; and different types of phosphate binders (e.g., a lanthanum-based binder could be combined with a calcium or aluminum-based binder).

Where the pharmaceutically active compound is an ACE inhibitor, it is typically enalapril or bezapril (FORTEKOR®); typical beta blockers include atenolol and propranolol; and amlodipine is a calcium channel blocker of choice.

The combination compositions of the present invention are benefited by the physical and chemical properties of the phosphate binders. Where the phosphate binder is a lanthanum oxycarbonate, for instance, it has the following properties: it is stable at high temperatures; it has a very high surface area; it is a white powder that can be granulated; it is sparingly soluble in aqueous media at pH above 2.0; and, it can be added directly to food and therefore does not need further formulation as a tablet.

The phosphate binder and other pharmaceutically active ingredient may be combined in any suitable way. Typically, however, the pharmaceutically active ingredient is adsorbed on or entrained within the phosphate binder.

The chemical and physical properties of the phosphate binders, especially, lanthanum oxycarbonate or lanthanum carbonate hydroxide, further aid in the formulation and stability of the combination product. For instance, pharmaceutically active ingredients (e.g., antihypertensives) undergo little degradation after binding to or entrainment within the phosphate binder. Typically, the pharmaceutically active ingredients undergo less than 10% degradation over a period of 1 month. Oftentimes, the active ingredient undergoes less than 5% or 2.5% degradation over a period of 1 month. In certain cases, the active ingredient undergoes less than 1.5% or 1% degradation over a period of 1 month.

Kits of the present invention typically include a container (e.g., bag, jar, can, etc.) of a composition comprising a lanthanum binding compound and another pharmaceutically active compound and instructions related to how the compound should be administered to a domestic animal. Information such as the amount of composition to be administered, and the regimen for administration is typically included on the instructions.

The methods of the present invention generally include the following step: administering a composition of the present invention to a domestic animals.

The compositions and methods of the present invention manage age-related diseases (e.g., CRF) in domestic animals. Exemplary domestic animals include dogs, cats, horses, rabbits, cows, goats and pigs. The present invention is particularly directed to the treatment of dogs, cats and horses.

Furthermore, in certain cases the compositions and methods delay the onset of age-related diseases (e.g., CRF) in domestic animals. In a side-by-side comparison, for instance, the compositions and methods of the present invention oftentimes provide for a statistically significant, beneficial difference from control in regard to onset of age-related diseases such as CRF. In certain cases, age-related diseases such as CRF may be delayed at least 1, 2 or 3 months over control.

As discussed above, the compositions and methods of the present invention ameliorate, or delay the onset of, age-related diseases. Moreover, the combination of phosphate binder and other pharmaceutically active compound provides for a synergistic effect. The compositions and methods, in other words, provide for a beneficial effect (e.g., CRF amelioration or delay of disease progression) that is more than one of ordinary skill in the art would expect from the addition of the compound to a phosphate binder.

Typically, the achieved synergism is at least 2.5% greater than the expected additive effect. Oftentimes the achieved synergism is at least 5% or 7.5% greater. In certain cases, the effect is 10% or 15% greater than the expected additive effect.

When lanthanum oxycarbonate is administered as the phosphate binder, the amount of phosphate binder in the combination composition administered to the domestic animal during a single administration typically ranges from 1.0 to 100 mg/kg body weight. Oftentimes the amount ranges from 30.0 to 80 mg/kg body weight. In certain cases the amount of administered lanthanum oxycarbonate ranges from 40.0 to 75.0 mg/kg body weight.

### EXAMPLES

### Example 1

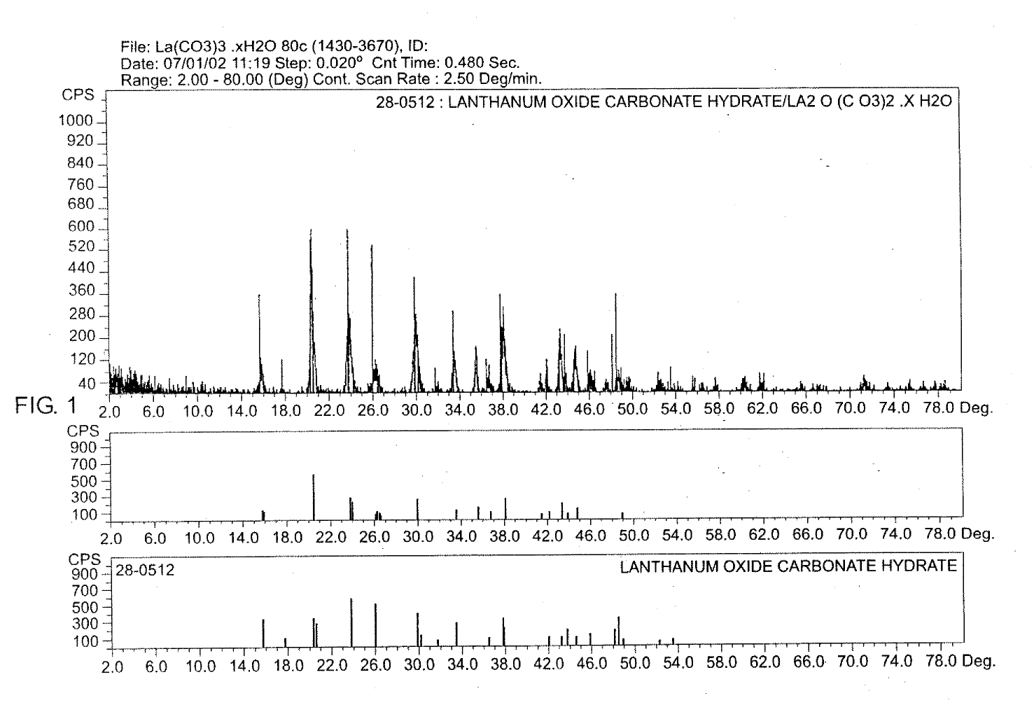

An aqueous HCl solution having a volume of 334.75 ml and containing LaCl₃ (lanthanum chloride) at a concentration of 29.2 wt % as La₂O₃ was added to a four liter beaker and heated to 80° C. with stirring. The initial pH of the LaCl₃ solution was 2.2. Two hundred and sixty five ml of an aqueous solution containing 63.59 g of sodium carbonate (Na₂CO₃) was metered into the heated beaker using a small pump at a steady flow rate for 2 hours. Using a Buchner filtering apparatus fitted with filter paper, the filtrate was separated from the white powder product. The filter cake was mixed four times with 2 liters of distilled water and filtered to wash away the NaCl formed during the reaction. The washed filter cake was placed into a convection oven set at 105° C. for 2 hours, or until a stable weight was observed. The product consists of lanthanum carbonate hydroxide, LaCO₃OH. FIG. 1 shows an X-ray diffraction scan of the compound as compared to a reference sample.

To determine the reactivity of the lanthanum compound with respect to phosphate, the following test was conducted. A stock solution containing 13.75 g/l of anhydrous Na₂HPO₄ and 8.5 g/l of HCl was prepared. The stock solution was adjusted to pH 3 by the addition of concentrated HCl. An amount of 100 ml of the stock solution was placed in a beaker with a stirring bar. Lanthanum oxycarbonate hydrate powder made as described above was added to the solution. The amount of lanthanum oxycarbonate hydrate powder was such that the amount of La in suspension was 3 times the stoichiometric amount needed to react completely with the phosphate. Samples of the suspension were taken at time intervals through a filter that separated all solids from the liquid. The liquid sample was analyzed for phosphorous.

### Example 2

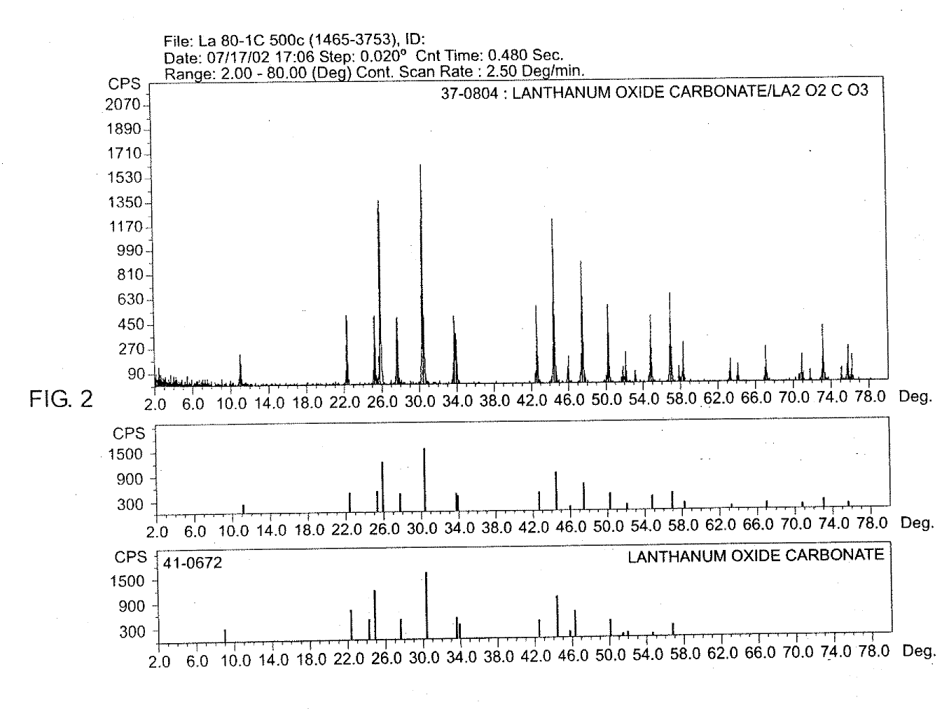

An aqueous HCl solution having a volume of 334.75 ml and containing LaCl₃ (lanthanum chloride) at a concentration of 29.2 wt % as La₂O₃ was added to a 4 liter beaker and heated to 80° C. with stirring. The initial pH of the LaCl₃ solution was 2.2. Two hundred and sixty five ml of an aqueous solution containing 63.59 g of sodium carbonate (Na₂CO₃) was metered into the heated beaker using a small pump at a steady flow rate for 2 hours. Using a Buchner filtering apparatus fitted with filter paper the filtrate was separated from the white powder product. The filter cake was mixed four times with 2 liters of distilled water and filtered to wash away the NaCl formed during the reaction. The washed filter cake was placed into a convection oven set at 105° C. for 2 hours until a stable weight was observed. Finally, the lanthanum oxycarbonate was placed in an alumina tray in a muffle furnace. The furnace temperature was ramped to 500° C. and held at that temperature for 3 hours. The resultant product was determined to be anhydrous lanthanum oxycarbonate La₂O₂CO₃. FIG. 2 shows an X-ray diffraction scan of the compound as compared to a reference standard.

The process was repeated three times. In one case, the surface area of the white powder was determined to be 26.95 m²/gm. A micrograph shows that the structure in this compound is made of equidimensional or approximately round particles of about 100 nm in size. An X-ray diffraction pattern showed that the product made is an anhydrous lanthanum oxycarbonate written as La₂O₂CO₃.

To determine the reactivity of this lanthanum compound with respect to phosphate, the following test was conducted. A stock solution containing 13.75 g/l of anhydrous Na₂HPO₄ and 8.5 g/l of HCl was prepared. The stock solution was adjusted to pH 3 by the addition of concentrated HCl. An amount of 100 ml of the stock solution was placed in a beaker with a stirring bar. Anhydrous lanthanum oxycarbonate made as described above, was added to the solution. The amount of anhydrous lanthanum oxycarbonate was such that the amount of La in suspension was 3 times the stoichiometric amount needed to react completely with the phosphate. Samples of the suspension were taken at intervals, through a filter that separated all solids from the liquid.

### Example 3

A solution containing 100 g/l of La as lanthanum acetate is injected in a spray-drier with an outlet temperature of 250° C. The intermediate product corresponding to the spray-drying step is recovered in a bag filter. This intermediate product is calcined at 600° C. for 4 hours. X-Ray diffraction of the product showed that it consists of anhydrous lanthanum oxycarbonate. The formula for this compound is written as (La₂CO₅).

To determine the reactivity of the lanthanum compound with respect to phosphate, the following test was conducted. A stock solution containing 13.75 g/l of anhydrous Na₂HPO₄ and 8.5 g/l of HCl was prepared. The stock solution was adjusted to pH 3 by the addition of concentrated HCl. An amount of 100 ml of the stock solution was placed in a beaker with a stirring bar. La₂CO₅ powder, made as described above, was added to the solution. The amount of lanthanum oxycarbonate was such that the amount of La in suspension was 3 times the stoichiometric amount needed to react completely with the phosphate. Samples of the suspension were taken at intervals through a filter that separated all solids from the liquid. The liquid sample was analyzed for phosphorous.

### Example 4

An aqueous HCl solution having a volume of 334.75 ml and containing LaCl₃ (lanthanum chloride) at a concentration of 29.2 wt % as La₂O₃ was added to a 4 liter beaker and heated to 80° C. with stirring. The initial pH of the LaCl₃ solution was 2.2. Two hundred and sixty five ml of an aqueous solution containing 63.59 g of sodium carbonate (Na₂CO₃) was metered into the heated beaker using a small pump at a steady flow rate for 2 hours. Using a Buchner filtering apparatus fitted with filter paper the filtrate was separated from the white powder product. The filter cake was mixed four times, each with 2 liters of distilled water and filtered to wash away the NaCl formed during the reaction. The washed filter cake was placed into a convection oven set at 105° C. for 2 hours or until a stable weight was observed. The X-Ray diffraction pattern of the product showed that it consists of lanthanum carbonate hydroxide, LaCO₃OH. The surface area of the product was determined by the BET method.

### Example 5

### In Vivo Study in Rats

Groups of six adult Sprague-Dawley rats underwent ⅚th nephrectomy in two stages over a period of 2 weeks and were then allowed to recover for a further two weeks prior to being randomized for treatment. The groups received vehicle (0.5% w/v carboxymethyl cellulose), or lanthanum oxycarbonate suspended in vehicle, once daily for 14 days by oral lavage (10 ml/kg/day). The dose delivered 314 mg elemental lanthanum/kg/day. Dosing was carried out immediately before the dark (feeding) cycle on each day. Urine samples (24 hours) were collected prior to surgery, prior to the commencement of treatment, and twice weekly during the treatment period. Volume and phosphorus concentration were measured.

Feeding—During the acclimatization and surgery period, the animals were given Teklad phosphate sufficient diet (0.5% Ca, 0.3% P; Teklad No. TD85343), ad libitum. At the beginning of the treatment period, animals were pair fed based upon the average food consumption of the vehicle-treated animals the previous week.

⅚ Nephrectomy—After one week of acclimatization, all animals were subjected to ⅚ nephrectomy surgery. The surgery was performed in two stages. First, the two lower branches of the left renal artery were ligated. One week later, a right nephrectomy was performed. Prior to each surgery, animals were anesthetized with an intra-peritoneal injection of ketamine/xylazine mixture (Ketaject a 100 mg/ml and Xylaject at 20 mg/ml) administered at 10 ml/kg, After each surgery, 0.25 mg/kg Buprenorphine was administered for relief of post-surgical pain. After surgery, animals were allowed to stabilize for 2 weeks to beginning treatment.

Results show a decrease in phosphorus excretion, a marker of dietary phosphorus binding, after administration of the lanthanum oxycarbonate or lanthanum carbonate hydroxide (at time>0), compared to untreated rats.

### Example 6

### Dog Study

Six adult beagle dogs were dosed orally with capsules of lanthanum oxycarbonate LaCO₃OH (compound A) or La₂O₂CO₃ (compound B) in a cross-over design using a dose of 2250 mg elemental lanthanum twice daily (6 hours apart). The doses were administered 30 minutes after provision of food to the animals. At least 14 days washout was allowed between the crossover arms. Plasma was obtained pre-dose and 1.5, 3, 6, 7.5, 9, 12, 24, 36, 48, 60, and 72 hours after dosing and analyzed for lanthanum using ICP-MS. Urine was collected by catheterization before and approximately 24 hours after dosing and creatinine and phosphorus concentrations measured. The tests led to reduction of urine phosphate excretion, a marker of phosphorous binding.

### Example 7

### Palatability Studies

Lanthanum oxycarbonate was mixed with wet and dry dog food and presented to 9 different dogs, almost all over 40 pounds. Each of the dogs ate the mixture, although 2 hesitated for some hours before eating. None of the dogs exhibited signs of nausea, vomiting, bloating or flatus during hours post meal.

Lanthanum oxycarbonate was mixed with cat food and presented to 2 cats, both old and one overweight. The first cat ate the food mixture. The second, which was the overweight cat, did not eat the mixture.

1. A composition comprising a phosphate binder and another pharmaceutically active ingredient, wherein the other pharmaceutically active ingredient is selected from a group consisting of antihypertensives, calcitrol, vitamin D analogues, lipid restriction products, potassium salts, anemia treatments, and alkalization compounds.

2. The composition according to claim 1, wherein the phosphate binder is selected from a group consisting of rare earth compounds, hydrophilic anion exchange resins, calcium salts, and aluminum salts.

3. The composition according to claim 1, wherein the other pharmaceutically active ingredient is an antihypertensive, and wherein the antihypertensive is selected from a group consisting of ACE inhibitors, beta blockers and calcium channel blockers.

4. The composition according to claim 2, wherein the phosphate binder is a rare earth compound, and wherein the rare earth compound is a lanthanum oxycarbonate or a lanthanum carbonate.

5. The composition according to claim 2, wherein the phosphate binder is a hydrophilic anion exchange resin, and wherein the resin is an aliphatic amine polymer.

6. The composition according to claim 2, wherein the phosphate binder is a calcium salt, and wherein the calcium salt is calcium acetate or calcium carbonate.

7. The composition according to claim 2, wherein the phosphate binder is an aluminum salt, and wherein the aluminum salt is aluminum hydroxide.

8. The composition according to claim 3, wherein the compound is an ACE inhibitor, and wherein the compound is enalapril or benzapril.

9. The composition according to claim 3, wherein the compound is a beta blocker, and wherein the beta blocker is atenolol or propranolol.

10. The composition according to claim 3, wherein the compound is a calcium channel blocker, and wherein the calcium channel blocker is amlodipine.

11. The composition according to claim 3, wherein the compound is a treatment for anemia such as Epogen®.

12. A kit for managing an age-related disease in a domestic animal, wherein the kit comprises:
a) a container, wherein the container includes a composition, and wherein the composition comprises a phosphate binder and another pharmaceutically active ingredient, and wherein the other pharmaceutically active ingredient is selected from a group consisting of antihypertensives, calcitrol, vitamin D analogues, lipid restriction products, potassium salts, treatments for anemia and alkalization compounds; and,
b) instructions related to how the composition should be administered to domestic animals.

13. The kit according to claim 12, wherein the phosphate binder is a rare earth compound.

14. The kit according to claim 13, wherein the other pharmaceutically active ingredient is an antihypertensive, and wherein the antihypertensive is selected from a group consisting of ace inhibitors, beta blockers and calcium channel blockers.

15. The kit according to claim 14, wherein the rare earth compound is a lanthanum oxycarbonate.

16. The kit according to claim 15, wherein the other pharmaceutically active ingredient is selected from a group consisting of enalapril, benzapril, atenolol, propranolol, and amlodipine.

17. A method of managing an age-related disease in a domestic animal, wherein the method comprises the steps of administering a composition to a domestic animal, wherein the composition comprises a phosphate binder and another pharmaceutically active ingredient, and wherein the other pharmaceutically active ingredient is selected from a group consisting of antihypertensives, calcitrol, vitamin D analogues, lipid restriction products, potassium salts, and alkalization compounds.

18. The method according to claim 17, wherein the phosphate binder is a rare earth compound.

19. The method according to claim 18, wherein the other pharmaceutically active ingredient is an antihypertensive, and wherein the antihypertensive is selected from a group consisting of ACE inhibitors, beta blockers and calcium channel blockers.

20. The method according to claim 19, wherein the rare earth compound is a lanthanum oxycarbonate.

21. The method according to claim 20, wherein the rare earth compound is a lanthanum oxycarbonate.
